(19) United States
(12) Plant Patent Application Publication
(10) Pub. No.: US 2007/0016988 P1
(43) Pub. Date: Jan. 18, 2007

(54) Grape plant named 'Arrafourteen'

(76) Inventors: Spiegel-Roy Pinhas, Ramat Gan (IL); Baron Izhak, Rehovot (IL); Sahar Hachman, Rehovot (IL); Perl Avihai, Rishon LeZion (IL)

Correspondence Address: CHRISTIE, PARKER & HALE, LLP, PO BOX 7068, PASADENA, CA, 91109-7068, US

(21) Appl. No.: 11/413,410

(22) Filed: Apr. 28, 2006

(30) Foreign Application Priority Data
Apr. 29, 2005 (ZA) VL 2005/6339

(51) Int. Cl. A01H 5/00

(52) U.S. Cl. PLT/205

(57) ABSTRACT

A new and distinct variety of Grapevine, designated ‘Arrafourteen’, characterized by strong vegetation, on own root and when grafted, red berry color with very uniform coverage and color, somewhat smaller bunches, and very juicy berries with pleasant taste and aroma.

### CROSS-REFERENCE TO RELATED APPLICATIONS

This application claims priority to South African PBR Registration Application No. VL 2005/6339, filed Apr. 29, 2005, the disclosure of which is incorporated herein by reference.

### BOTANICAL CLASSIFICATION/CULTIVAR DENOMINATION

Vitis sp. cultivar Arrafourteen.

### BACKGROUND OF THE INVENTION

The present invention relates to a new and distinct variety of Grapevine, botanically known as Vitis sp., and hereinafter referred to by the name ‘Arrafourteen’.

### SUMMARY OF THE INVENTION

‘Arrafourteen’ was discovered as a red fruit mutation amount the parent ‘Prime 125’ seedless cultivar.

It has been asexually reproduced at Nancy Farms in Paarl, South Africa, by budwood vegetative propagation. Observations made on ‘Arrafourteen’ since 2001 have shown that the unique features of this new Grapevine are stable and reproduced true to type in successive generations.

The new variety differs from other cultivars known to the inventor, including its parents, in the following traits, which have been repeatedly observed and are determined to be the unique and stable characteristics of ‘Arrafourteen’:
1. Strong vegetation variety, on own root and when grafted;
2. Produces small bunches compared to parent Prime Seedless;
3. Berry color is red, in comparison with the green of the Prime Seedless;
4. Ripening time is 2-3 days later compared to the green Prime Seedless;
5. Midseason variety, ripening in the last half of June.

These characteristics in combination distinguish ‘Arrafourteen’ as a new and distinct Grapevine cultivar. All observations were made from four year old specimens grown in Paarl, South Africa, from 2001 till 2005.

### BRIEF DESCRIPTION OF ILLUSTRATIONS

The accompanying photographic illustrations show typical specimens of four year old specimens of the new variety, in color as nearly true as it is reasonably possible to make in a color illustration of this character. Colors in the photograph may differ from the color values cited in the detailed botanical description below, which accurately describes the colors of the new Grapevine.

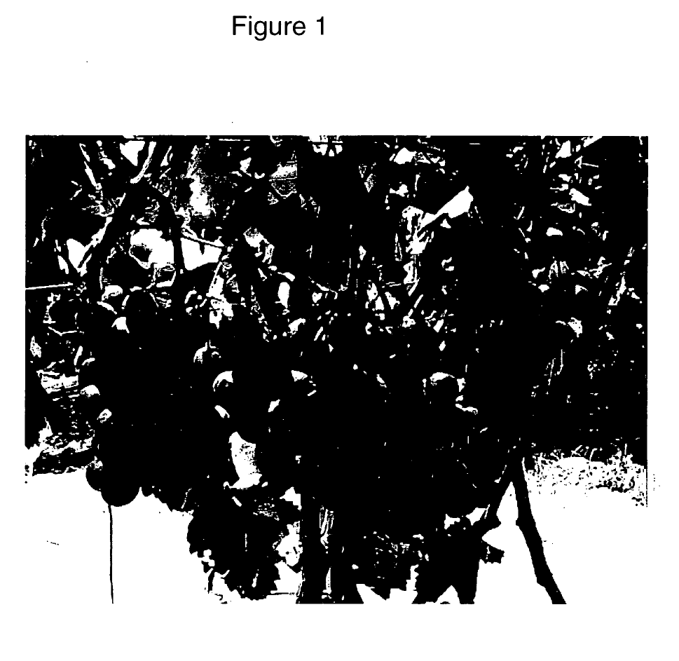

FIG. 1 comprises leaves, stems and grapes of ‘Arrafourteen’.

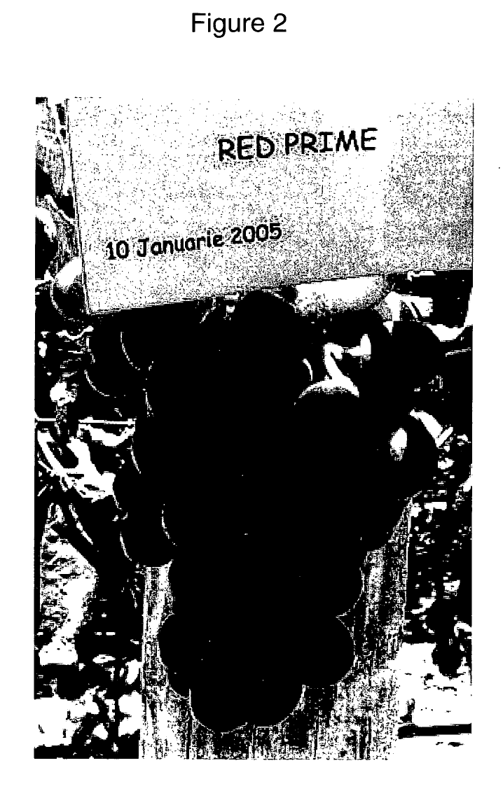

FIG. 2 comprises a close-up of a bunch of ‘Arrafourteen’.

### DESCRIPTION OF THE NEW VARIETY

Referring now specifically to the new and distinct variety of Grapevine, the following is a detailed description of ‘Arrafourteen’ with color description where indicated by reference to The Royal Horticultural Society Colour Chart, except where common terms of color definition are employed.

The new variety of Grapevine as herein described may vary in slight detail due to climatic, soil and cultural conditions under which the variety may be grown, the present description being of four year old specimens of the variety as grown from its own root at Paarl, South Africa, between 2001 and January of 2005.
Plant characteristic:
   Form.—Upright with canopy of four feet on either side of the trunk, vines of 157 cm to 197 cm in width perpendicular to a row.
   Growth.—low vigor, particularly weak during flowering.
Vegetative characteristics:
   Trunk.—size — medium developed for age, ranges from 46 mm to 88 mm. surface texture — rough. color — Greyed Green near group 197.
   Canes.—form of growing tip — half opened. cross-sectional form — circular to slightly eliptical. thickness — small to medium from 8 mm to 17 mm diameter. average surface texture — relatively smooth with occasional ribbing. length — slightly less than average with an average joint frequency of 60 mm to 160 mm. shoot color — Yellow Green Group near 153A, intermixed with bands of Orange-Red near group 34. form of tendrils — predominantly trifid, with occasional bifid. distribution of tendrils — discontinuous. length of tendrils — average length 11.5 cm to 18 cm.
   Leaf.—form — generally pentagonal. shape of blade — kidney shape with five lobes. marginal form — broadly serrate. length of leaf — average mature leaf ranges from 115 to 152 mm as measured from petiolar junction to apex of central lobe. width of leaf — average widty 56 mm to 94 mm. surface texture — relatively smooth. top color — Green Group near 143A. bottom color — Yellow-Green Group near 145A. stick color — Yellow-Green Group near 152A. anthocyanin coloration on main veins on upper side of blade (Recorded on leaves above the cluster at the medium third of shoot) — Yellow-Green Group near 144C. profile (cross-section at the middle of the leaf blade) — flat. lobe apices form — acute shape of teeth (Recorded on the lateral lobe) — Both sides convex. length of teeth: largest range from 2 to 4 mm. ratio length/width of teeth — Medium. thickness vein — average. general shape of petiole sinus (Degree of the opening of the petiole sinus) — distinctly “V” shaped with lobes overlapping with no particularities of petiole sinus. tooth of petiole sinus — both sides convex and medium in length. length of petiole to middle vein — equal. length petiole — average length 78 mm to 109 mm. thickness petiole — varies from 2 to 3 mm. stipules — absent.
Inflorescence and fruit:
   Inflorescenc.—sex of flower — hermaphroditic medium length.
   Flower.—form — cluster of small to medium size. location — first floral cluster occurs from third to sixth joint. length — bloom ranges from 30 mm to 35 mm from base of peduncle to tendril and from 18 to 30 mm from tendril to first rachis branch. floral cluster length — ranges from 11 to 18.5 cm without peduncle. floral cluster width — ranges from 6 to 12 cm. stamens form — upright. anthers size — average. pollen quantity — abundant. calyptra — separates completely from base of flower.
   Bunch.—size (without peduncle) — length ranges from 14 to 22.8 cm, width ranges from 10 to 15 cm. density — slight (pedicles not visible) length of peduncle — average length 18 to 44 mm, thickness 2.5 to 4.5 mm. shape — conical. number of berries — approximately 70 to 110 berries per bunch. color of peduncle — Yellow-Green Group near 144C.
   Berry.—size — diameter ranges from 18 to 24 mm, length ranges from 19 to 24 mm. shape — cylindrical in lateral aspect, berry cross-section is circular. presence of seed — absent. skin color — Red Group near 44A, uniform even under shade, hilum visible. skin texture — medium thick skin. weight — 10 berries, about 47 g. firmness of flesh (Weight necessary for cracking the berries) — soft. juiciness of flesh — juicy. aroma — special, pleasant. particular flavor — sweet and well-balanced. pedicel size — length ranges from 4 to 11 mm, thickness ranges from 1.5 to 2.8 mm at mid pedicel. ease of detachment from pedicel — easy.
   Plant descriptors.— All information below based on growing the variety in the South Africa.
   Time of bud burst (only varieties for fruit production).—In the first week of November.
   Inflorescence.—cluster of small to medium size.
   Time of berry ripening (veraison).—early January.
   Bunch length (without peduncle).—length ranges from 14 to 22.8 cm.
   Berry thickness of skin (thickness of epidermis plus hypodermis).—medium thick.
   Market use information.—fresh table grape, high quality seedless for fresh consumption, shipping quality of the fruit is very good both in storage and transportation stem retains turgidity for extended period.
   Resistance/susceptibility to disease and pest.—No difference from other Vitis Venifera varieties.
Comparison to flame seedless: Prime seedless is a popular green grape in the USA. It is early, crunchy and pleasant to eat. Prime seedless is different from the new variety by the following: ‘Arrafourteen’ ripening time is two to three days later than Prime seedless; ‘Arrafourteen’ has a smaller bunch size than green Prime seedless; and ‘Arrafourteen’ has a uniform red color fully covered from early stages of ripening, while Prime seedless are green.

We claim:

1. A new and distinct variety of grapevine, botanically known as Vitis sp., identified as ‘Arrafourteen’, substantially as shown and described herein.
